(19) United States
(12) Patent Application Publication
(10) Pub. No.: US 2007/0248556 A1
(43) Pub. Date: Oct. 25, 2007

(54) Administration of C-glycoside compounds for activating and regulating cutaneous immunity

(75) Inventors: Nathalie Pineau, Herblay (FR); Maria Dalko, Gif S/Yvette (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/783,411

(22) Filed: Apr. 9, 2007

Related U.S. Application Data
(60) Provisional application No. 60/797,390, filed on May 4, 2006.

(30) Foreign Application Priority Data
Apr. 7, 2006 (FR) 0651265

(51) Int. Cl. A61K 8/73; A61K 31/70

(52) U.S. Cl. 424/70.13; 514/25

(57) ABSTRACT

C-glycoside compounds having the general formula (1): 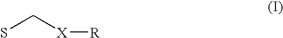 are useful for stimulating the immune system of the skin and/or as immunoregulators, and are advantageously formulated into cosmetic/pharmaceutical compositions suited for preventing and/or limiting cutaneous immune imbalances, notably those resulting from environmental stresses.

### CROSS-REFERENCE TO PRIORITY/PROVISIONAL APPLICATIONS

This application claims priority under 35 U.S.C. § 119 of FR 06/51265, filed Apr. 7, 2006, and of U.S. Provisional Application No. 60/797,390, filed May 4, 2006, each hereby expressly incorporated by reference and each assigned to the assignee hereof.

### CROSS-REFERENCE TO COMPANION APPLICATION

Copending U.S. Patent Application No. ______ [Attorney Docket No. 1016800-000847], filed concurrently herewith, and also expressly incorporated by reference and assigned to the assignee hereof.

### BACKGROUND OF THE INVENTION

1. Technical Field of the Invention

The present invention relates to the administration of C-glycoside compounds as agents for stimulating the immune system of the skin and/or as immunoregulators, and for formulating compositions containing a cosmetically or pharmaceutically acceptable medium, useful in particular to prevent and/or limit the appearance of cutaneous immune imbalances, in particular related to environmental stresses.

2. Description of Background and/or Related and/or Prior Art

Cutaneous immune disorders are normal physiological phenomena which appear with age. They can, however, be accelerated by infections with microorganisms (viruses and bacteria), stress, chronological aging, ultraviolet rays, “urban” living conditions, etc.

The immune system comprises a collection of specialized cells which are subject to multiple control mechanisms that ensure their renewal, their activation and their differentiation, and are essential to a normal level of immunocompetence. The role of the immune system is to discriminate self from non-self in order to eliminate pathogenic agents and spontaneous tumors. Any cellular depletion, any incorrect immune regulation or any functional deficiency is liable to promote the occurrence of manifestations which range from discomfort to pat hological disorders characterized by the disturbance of the mechanisms of recognition of self with respect to non-self, and a greater sensitivity with respect to microbial attacks and neoplastic processes.

The skin is an organ that is highly important for the organism and is recognized as one of the main active elements of the immune defense system. Three epidermal cell types participate in this system: keratinocytes, melanocytes and Langerhans cells. These cells, which are found only in the skin, play an essential role in the immune response, and in particular in antigen presentation.

Normal skin constitutes a barrier and is capable of defending itself against outside attacks, in particular chemical and mechanical attacks; in this respect, a certain number of defense reactions against environmental factors (climate, ultraviolet rays, tobacco, pollutants, etc.) and/or xenobiotics (such as, for example, certain medicaments) occur therein.

Various factors, such as atmospheric pollutants, detergents, allergens, UV radiation, etc., negatively affect, by virtue of their action on the skin, a variety of immune responses, both locally at the site of exposure, and systemically, at distant sites. This form of immunosuppression is in particular related to the induction of antigen-specific suppressor T cells. Impairment of the delayed response is particularly important since immune reactions generated by T lymphocytes are responsible for protection against many chronic infectious pathologies.

Pathologies also exist which are based not on an insufficiency of immune cells, but on an immune imbalance; this is the case, in particular, of atopic diseases and autoimmune diseases which present, respectively, an excess of Th-2 lymphocytes and an excess of Th-1 lymphocytes.

The prevalence of atopic diseases (accompanied by an excessive presence of Th-2-type lymphocytes, such as atopic dermatitis, gastrointestinal allergies, allergic rhinitis and conjunctivitis, asthma) and of autoimmune diseases (accompanied by an excessive presence of Th-1-type lymphocytes, such as psoriasis, vitiligo, diffuse scleroderma, lupus erythematosus, certain forms of alopecia, rheumatoid arthritis, type I diabetes) has gradually increased over the last few decades in western societies.

The explanation which has appeared to be the most plausible regarding the increase in Th-2-related conditions is the hygiene-related hypothesis which suggests that the rapid increase in atopic eczemas is related to the current cleanliness of environments and to the decrease in exposure to microorganisms at the beginning of life (Holt PG, in Nestle Nutrition Workshop series Pediatric Program, Isolauri E. et al., ed, Allergic diseases and the environment, Karger AG, Basel, vol. 53 pp 53-68, 2004).

During the allergic reaction, which can be explained by a reorientation of Th-1-type immune reactions towards Th-2-type responses, the interaction from the normal host and the allergen is altered. The allergic reaction is then accompanied by an imbalance in the immune response, which may then be induced by resident bacteria (Martinez FD, Respir. Res., 2:129-132, 2001).

These atopic conditions are chronic and often systemic inflammatory reactions of complex origin (genetic and environmental factors). In these pathologies, the responses of type 2 (Th-2) T helper cells to “inoffensive” antigens (allergens) of the environment play a determining role in triggering allergic conditions (Romagnani S, Curr. Opin. Immunol., 6:838-846,1994). Th-2 cells explain the joint intervention, in the allergic inflammatory process, of B cells that produce E immunoglobulins (via the production of interleukins IL-4 and IL-13), and of mast cells (via the production of IL-5).

Moreover, it is also important to emphasize that, while there is currently an increase in allergic pathologies related to Th-2-type cells, at the same time, an increase in pathologies related to Th-1 cells (autoimmune diseases, such as type I diabetes, psoriasis or vitiligo) is observed in developing countries.

Th-1-type cells play an important role in the development of the delayed hypersensitivity reaction (DHR); thus, in certain chronic autoimmune diseases such as rheumatoid arthritis and thyroiditis, the skin lesions observed are of the DHR type, and the CD4 T cells within said lesions are mainly of the Th-1 type. Identical results have been obtained over the course of infectious diseases due to mycobacteria (tuberculosis, leprosy), over the course of Lyme disease and over the course of psoriasis.

Vitiligo is an acquired depigmentation disorder of the skin that affects 1% of the world's population, regardless of skin color. Vitiligo is a skin disease in which the melanocytes (MCs) are eliminated from the basal layer of the epidermis in the lesions. This disappearance of melanocytes leads to a deficient pigmentation. In vitiligo lesions, the melanocytes are destroyed by MC-reactive T cells. The depigmentation frequently begins during adolescence.

For example, after an infection, UV radiation or a chemical/mechanical attack, the melanocytes are damaged. Under conditions of normal immune control, these impairments are controlled by the immune system which destroys the modified cells. In the case of vitiligo, these impairments are not correctly treated and they constitute a source of auto-antibodies which will contribute to establishing the autoimmune pathology.

Thus, it appears that a therapy which would make it possible to reorient the Th-2 “allergic” or Th-1 “autoimmune” immune response towards a physiological balance would lead to products whose topical application could induce a regulation of local immune phenomena.

The assignee hereof has now demonstrated that C-glycoside compounds of general formula (I) are capable both of stimulating the immune system of the skin and also of rectifying an immune imbalance from populations of Th-1 and Th-2 lymphocytes, and are capable of causing atopic or autoimmune disorders.

It is known that certain sugars such as aldoses, ketoses, deoxyoses or monosaccharide derivatives stimulate immune defenses (EP-0,818,201).

O-glycoside or C-glycoside molecules which modulate the immune system also exist, such as C-glycolipid compounds (WO 2003/105769), fucopeptides and amido-deoxygalactose derivatives (U.S. Pat. No. 5,962,660 and WO 96/29339).

### SUMMARY OF THE INVENTION

The present invention features the administration of compounds of general formula (I): 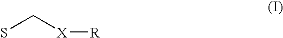
in which,
S is a monosaccharide or a polysaccharide containing up to 20 sugar units, in pyranose and/or furanose form, and of the L and/or D configuration, said mono- or polysaccharide having at least one hydroxyl function that is necessarily free and/or optionally one or more optionally protected amine functions;
the S—CH₂X bond is a bond of C-anomeric nature;
X is a group selected from: —CO—, —CH(NR₁R₂)—, —CHR′— and —C(═CHR′)—;
R is a linear or branched, saturated or unsaturated alkyl, perfluoroalkyl or hydrofluoroalkyl chain, or a cycloalkyl, cycloperfluoroalkyl or cyclohydrofluoroalkyl ring member having from 1 to 18 carbon atoms, or a phenyl radical, the said chain, said ring member or said phenyl radical optionally being interrupted with one or more heteroatoms selected from among oxygen, sulfur, nitrogen and silicon, and optionally substituted with at least one radical selected from among —OR′₁—, —SR″₁, —NR′″₁R′₂, —COOR″₂, —CONHR′″₂, —CN, halogen, perfluoroalkyl and hydrofluoroalkyl, and/or at least one optionally substituted cycloalkyl, aryl or heterocyclic radical;
R′, R₁ and R₂, which may be identical or different, have the same definition as that given for R, and can also represent a hydrogen and a hydroxyl radical;
R′₂ and R′″₂, which may be identical or different, represent a hydrogen atom, or a radical selected from among a hydroxyl radical and a linear or branched, saturated or unsaturated alkyl, perfluoroalkyl and/or hydrofluoroalkyl radical having from 1 to 20 carbon atoms;
R′₁, R″₁, R″₂ and R′″₁, which may be identical or different, represent a hydrogen atom, or a radical selected from among a linear or branched, saturated or unsaturated alkyl, perfluoroalkyl and/or hydrofluoroalkyl radical having from 1 to 20 carbon atoms;
   with the following provisos:
R₁ and R₂ cannot simultaneously each be a hydroxyl radical;
R′″₁ and R′₂ cannot simultaneously each be a hydroxyl radical;
when S is a D-xylose and X is ═CO, then R cannot be an unsubstituted phenyl;
when S is a D-glucose and X is the function —OH, then R cannot be a p-hydroxyphenyl; and
when S is a D-glucose and X is ═O, then R cannot be a p-methoxyphenyl or a hexyl;
   for combating the weakening of the natural defenses of the skin which appears during chronological or photoinduced aging and/or reinforcing the natural defenses of the skin.

### DETAILED DESCRIPTION OF BEST MODE AND SPECIFIC/PREFERRED EMBODIMENTS OF THE INVENTION

The C-glycoside compounds that are administered according to the invention, whether regime or regimen, represent a subfamily of the C-glycoside derivatives described in EP-1,345,919; they can be prepared according to the process described therein.

Preferably, the compounds administered according to the invention are such that:
S is a monosaccharide or a polysaccharide having up to 5 sugar units, in pyranose and/or furanose form, and of the L and/or D configuration, said mono- or polysaccharide having at least one hydroxyl function that must be free and/or optionally one or more optionally protected amine functions;
R is a linear or branched, saturated or unsaturated alkyl radical, or a cycloalkyl ring member, having from 1 to 14 carbon atoms, or a phenyl radical, said radical, said ring member or said phenyl radical optionally being substituted with at least one radical selected from among —OR′₁, —NR′″₁R′₂, —COOR′₂, —CONHR′″₂;
R′, R₁ and R₂, which may be identical or different, have the same definition as that given for R, and can also represent a hydrogen and a hydroxyl radical, with the proviso that R₁ and R₂ cannot simultaneously each be a hydroxyl radical;
R′₂ and R′″₂, which may be identical or different, represent a hydrogen atom, or a radical selected from among a hydroxyl radical and a linear or branched, saturated or unsaturated alkyl radical having from 1 to 8 carbon atoms, with the proviso that R′″₁ and R′₂ cannot simultaneously each be a hydroxyl radical;
R′₁, R″₁, R″₂ and R′″₁, which may be identical or different, represent a hydrogen atom, or a radical selected from among a linear or branched, saturated or unsaturated alkyl radical having from 1 to 8 carbon atoms;
   the definitions of the other substituents of general formula (I) remaining unchanged.

According to another embodiment, preference will be given to the compounds of general formula (I) such that:
S is a monosaccharide or a polysaccharide having up to 2 sugar units, in pyranose and/or furanose form, and of the L and/or D configuration, said mono- or polysaccharide having at least one hydroxyl function that must be free and/or optionally one or more optionally protected amine functions;
X is a group selected from: —CO—, —CH(NR₁R₂)— and —CHR′; the definitions of the other substituents of general formula (I) remaining unchanged or having the preferred values given above.

Advantageously, the preferred monosaccharides are selected from among D-glucose, D-mannose, D-xylose, D-lyxose, L-arabinose, L-rhamnose, D-glucuronic acid, D-galacturonic acid, D-iduronic acid, N-acetyl-D-glucosamine and N-acetyl-D-galactosamine, and more advantageously D-glucose, D-xylose, N-acetyl-D-glucosamine or L-fucose, and very preferably D-xylose.

More advantageously, the preferred polysaccharides having up to 6 sugar units are selected from among D-maltose, D-cellobiose, D-maltotriose, a disaccharide combining a uronic acid selected from D-iduronic acid or D-glucuronic acid, with a hexosamine selected from among D-galactosamine, D-glucosamine, N-acetyl-D-galactosamine and N-acetyl-D-glucosamine, and an oligosaccharide containing at least one xylose advantageously selected from among xylobiose, methyl-β-xylobioside, xylotriose, xylotetraose, xylopentaose and xylohexaose, and preferably xylobiose, which is composed of two xylose molecules linked by a 1,4-linkage.

Among the C-glycoside derivatives of formula (I) according to the invention, the following are most particularly preferred:

Compound 1. 1-(C-β-D-Xylopyranosyl)propan-2-one:

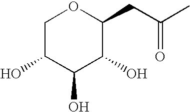

Compound 2. 1-(C-β-D-Glucopyranosyl)propan-2-one:

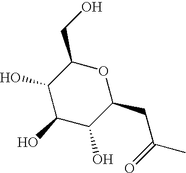

More particularly, the administration of the C-glycosides of general formulae (I) according to the invention is suitable for preparing the skin against exposure to the sun.

Thus, the present invention makes it possible to prevent and/or limit the harmful effects of exposure to UV rays.

The C-glycosides of general formulae (I) can also be administered for maintaining a balance from Th-1 and Th-2 lymphocyte populations and/or for correcting an immune imbalance related to an excess of Th-1-type lymphocytes or Th-2-type lymphocytes.

These compounds according to the invention may therefore be advantageously administered for combating undesirable manifestations of atopic type, in particular for treating reactive skin (characterized by red blotches, painful sensations, swelling), for preventing and/or decreasing itching, or else combating autoimmune conditions such as an imbalance in pigmentation of the skin and/or of the hair, in particular hair turning white or grey prematurely.

According to another of its embodiments, the present invention features formulating C-glycoside compounds of general formulae (I), into compositions comprising a physiologically acceptable medium, useful for the prevention and/or treatment of cutaneous autoimmune diseases or cutaneous atopic disorders.

More particularly, the cutaneous atopic disorders are selected from among cutaneous allergic reactions, atopic dermatitis and atopic eczema, and the cutaneous autoimmune diseases are selected from among delayed contact hypersensitivity, psoriasis, vitiligo, diffuse scleroderma, lupus erythematosus or certain forms of alopecia.

The term “immunostimulant agent” means a compound whose administration to an organism results in the proliferation of the immune cells of said organism, for example, the lymphocytes.

The term “immunoregulatory agent” means an agent capable of maintaining and/or reestablishing a cutaneous immune balance from Th-1-type and Th-2-type cell populations, or else of correcting an excessive presence of Th-1-type or Th-2-type cells.

An immune imbalance may in particular be demonstrated by virtue of the increase, in an organism, of one or more cytokines characteristic of a lymphocyte type.

In fact, in addition to their classification according to the structure of their T receptor, Th-1-type and Th-2-type lymphocytes have been classified according to their cytokine protocol.

The cytokines characteristic of type 1 (Th-1) lymphocytes are IL-2, IFN-γ and TNF-β. The cytokines of type 2 (Th-2) lymphocytes are IL-4, IL-5, IL-9, IL-10 and IL-13.

More generally, the C-glycoside compounds of general formulae (I) can be administered as an immunostimulant medicament in humans or in animals.

For this type of indication, the compositions comprising the C-glycoside compounds of general formulae (I) can be administered, for example, parenterally, (intraperitoneally, subcutaneously, intramuscularly, intravenously, percutaneously), orally, nasally, conjuctivally, rectally or perlingually.

They can also be administered by local application, for example, by means of orally disintegrating tablets, in particular in non-specific immunotherapy of oral cavity diseases.

The medicaments of the invention can be administered by way of prophylaxis, in the various cases above, and in particular for the prevention of recurring infections of the ear, nose and throat (ENT) sphere, and for the prevention of risks of infection in chronically ill patients.

The medicaments of the invention are administered in particular as an immunostimulant treatment, in the ENT or bronchopulmonary field (rhinopharyngitis, laryngitis, sinusitis, sore throats, otitis, bronchitis, etc.) or in the dermatological field, in the case of bacterial, fungal or viral infections.

Preferably, the C-glycoside compounds of general formulae (I) according to the invention will be formulated into a cosmetic or pharmaceutical composition intended to be applied topically to the skin, the scalp or the mucous membranes.

The compositions according to the invention can be in any of the forms suitable for the applications envisaged, in particular topical application, in the cosmetics and dermatological fields.

The compositions according to the invention contain a physiologically acceptable medium and one or more compounds according to the invention in an effective amount for stimulating the immunity of the skin or for re-equilibrating the balance from Th-1 and Th-2 lymphocytes, for example, in an amount ranging from 0.01% to 30% by weight, and preferably from 0. 1% to 5% by weight, relative to the total weight of the composition.

The term “physiologically acceptable medium” means a medium compatible with the skin and, optionally, with the mucous membranes, the nails, the scalp and/or the hair.

The compositions according to the invention can be in the form in particular of an aqueous solution or a dispersion of the lotion or serum type, emulsions with a liquid or semi-liquid consistency, of the milk type, obtained by dispersion of a fatty phase in an aqueous phase (ONV) or vice versa (W/O), or suspensions or emulsions which are soft in consistency, of the aqueous or anhydrous gel or cream type, or else microcapsules or microparticles, or vesicular dispersions of ionic and/or non-ionic type. These compositions are prepared according to the usual methods.

This composition may be more or less fluid and may have the appearance of a white or colored cream, an ointment, a milk, a lotion, a serum, a paste or a foam. It can optionally be applied to the skin in the form of an aerosol. It can also be in the form of a solid, for example, in the form of a stick. It can be used as a care product, as a cleansing product, as a makeup product or alternatively as a shampoo or conditioner.

When the composition according to the invention is an emulsion, the proportion of the fatty phase can range from 5% to 80% by weight, and preferably from 5% to 50% by weight, relative to the total weight of the composition. The oils, the waxes, the emulsifiers and the coemulsifiers used in the composition in the form of an emulsion are selected from those conventionally used in the cosmetics field. The emulsifier and the coemulsifier are present, in the composition, in a proportion ranging from 0.3% to 30% by weight, and preferably from 0.5% to 20% by weight, relative to the total weight of the composition. The emulsion may also contain lipid vesicles.

The compositions according to the invention are useful for a cosmetic or pharmaceutical, particularly dermatological, application. The compositions according to the invention are preferably suited for cosmetic applications.

The present invention therefore also features a cosmetic treatment regime or regimen for the skin or for the scalp, comprising the topical application to the skin or the scalp of the compositions described above.

Given the immunostimulant and equilibrating properties of the compounds according to the invention, this process is, in particular, useful to reinforce the natural defenses of the skin and to improve the cutaneous immune balance.

The C-glycoside compounds according to the invention will advantageously be combined with active agents for the hair, selected from among:
anti-seborrhoeic agents, such as certain sulfur-containing amino acids, 13-cis-retinoic acid or cyproterone acetate;
agents for combating squamous states of the scalp (dandruff), such as zinc pyrithione, selenium disulfide, climbazole, undecylenic acid, ketoconazole, piroctone olamine (octopirox) or cyclopiroctone (cyclopirox);
active agents for stimulating hair regrowth and/or promoting the slowing down of hair loss, particularly:
nicotinic acid esters, including in particular tocopheryl nicotinate, benzyl nicotinate and C₁-C₆ alkyl nicotinates, for example methyl nicotinate or hexyl nicotinate;
pyrimidine derivatives, such as 2,4-diamino-6-piperidinopyrimidine 3-oxide or “Minoxidil” described in U.S. Pat. Nos. 4,139,619 and 4,596,812; Aminexil or 2,4-diaminopyrimidine 3-oxide described in WO 96/09048;
agents that are both lipoxygenase inhibitors and cyclooxygenase inducers, or cyclooxygenase-inducing agents that promote hair regrowth, such as those described in EP-0,648,488;
antibiotics such as macrolides, pyranosides and tetracyclines, and in particular erythromycin;
cinnarizine, nimodipine and nifedipine;
hormones, such as estriol or the like, or thyroxine and salts thereof;
anti-androgenic agents, such as oxendolone, spironolactone, diethylstilbestrol and flutamide;
cromakalim and nicrorandil.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Demonstration of the Immunostimulant Activity of the C-Glycoside Derivatives of General Formula (I)

The immunostimulant activity is tested in the following way: human peripheral blood cells are cultured in the presence of an RPMI-type culture medium supplemented with L-glutamine (2 mM), penicillin/streptomycin (50 μg/50 IU/ml) and foetal calf serum (10%). The C-glycoside derivatives are added at various concentrations (10 to 0.05 mM), as is phytohaemagglutinin (PHA at 5 *G/ml), a positive control for lymphocyte proliferation. After 3 days of culture, the proliferation is revealed by BrdU labeling.

The results obtained are as follows:

| Active agent | % stimulation relative to the control | | | | | |
|---|---|---|---|---|---|---|
| 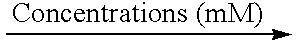 | 10 | 5 | 1 | 0.5 | 0.1 | 0.05 |
| Compound 1: 1-(C-β-D- | 74* | 106* | 131 | 84 | 115 | 108 |
| Xylopyranosyl)propan-2-one | | | | | | |
| Compound 2: 1-(C-β-D- | 224 | 227 | 123 | 90 | 76 | 64 |
| Glucopyranosyl)propan-2-one | | | | | | |

*cytotoxicity

All the derivatives tested exhibit a strong capacity for human lymphocyte proliferation.

Compounds 1 and 2 stimulate human lymphocyte proliferation from 1 mM, these compounds therefore have an immunostimulant activity.

### EXAMPLE 2

### Formulations

Face care gel:

| | |
|---|---|
| Compound 2 | 0.05% |
| Thickening polymer | 1.00% |
| Antioxidant | 0.05% |
| Isopropanol | 40.00% |
| Preservative | 0.30% |
| Water | qs 100% |

Face lotion for hyperreactive skin:

| | |
|---|---|
| Compound 1 | 0.50% |
| Magnesium gluconate | 3.00% |
| Antioxidant | 0.05% |
| Isopropanol | 40.0% |
| Preservative | 0.30% |
| Water | qs 100% |

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for combating the weakening of the natural defenses of the skin that occur as a result of chronological or photoinduced aging and/or for reinforcing the natural defenses of the skin, comprising administering to an individual in need of such treatment, a thus effective amount of at least one compound having the following general formula (I): 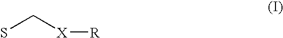
in which,
S is a monosaccharide or a polysaccharide containing up to 20 sugar units, in pyranose and/or furanose form, and of the L and/or D configuration, said mono- or polysaccharide having at least one hydroxyl function that is necessarily free and/or optionally one or more optionally protected amine functions;
the S—CH₂X bond is a bond of C-anomeric nature;
X is a group selected from: —CO—, —CH(NR₁R₂)—, —CHR′— and —C(═CHR′)—;
R is a linear or branched, saturated or unsaturated alkyl, perfluoroalkyl or hydrofluoroalkyl chain, or a cycloalkyl, cycloperfluoroalkyl or cyclohydrofluoroalkyl ring member having from 1 to 18 carbon atoms, or a phenyl radical, the said chain, said ring member or said phenyl radical optionally being interrupted with one or more heteroatoms selected from among oxygen, sulfur, nitrogen and silicon, and optionally substituted with at least one radical selected from among —OR′₁—, —SR″₁, —NR′″₁R′₂, —COOR″₂, —CONHR′″₂, —CN, halogen, perfluoroalkyl and hydrofluoroalkyl, and/or at least one optionally substituted cycloalkyl, aryl or heterocyclic radical;
R′, R₁ and R₂, which may be identical or different, have the same definition as that given for R, and can also represent a hydrogen and a hydroxyl radical;
R′₂ and R′″₂, which may be identical or different, represent a hydrogen atom, or a radical selected from among a hydroxyl radical and a linear or branched, saturated or unsaturated alkyl, perfluoroalkyl and/or hydrofluoroalkyl radical having from 1 to 20 carbon atoms;
R′₁, R″₁, R″₂ and R′″₁, which may be identical or different, represent a hydrogen atom, or a radical selected from among a linear or branched, saturated or unsaturated alkyl, perfluoroalkyl and/or hydrofluoroalkyl radical having from 1 to 20 carbon atoms;
with the following provisos:
R₁ and R₂ cannot simultaneously each be a hydroxyl radical;
R′″₁ and R′₂ cannot simultaneously each be a hydroxyl radical;
when S is a D-xylose and X is ═CO, then R cannot be an unsubstituted phenyl;
when S is a D-glucose and X is the function —OH, then R cannot be a p-hydroxyphenyl; and
when S is a D-glucose and X is ═O, then R cannot be a p-methoxyphenyl or a hexyl.

2. The regime or regimen as defined by claim 1, for preparing the skin against exposure to the sun and/or preventing and/or limiting the harmful effects of UV rays.

3. The regime or regimen as defined by claim 1, for treating reactive skin and/or preventing and/or decreasing itching.

4. The regime or regimen as defined by claim 1, for preventing and/or treating autoimmune disorders associated with an imbalance in pigmentation of the skin and/or of the hair.

5. The regime or regimen as defined by claim 1, for preventing and/or treating hair turning white or grey prematurely.

6. The regime or regimen as defined by claim 1, comprising co-administering at least one active agent for the hair.

7. A regime or regimen for the prevention and/or treatment of a cutaneous autoimmune disease state or a cutaneous atopic disorder, comprising administering to an individual in need of such treatment, a thus effective amount of at least one compound having the following general formula (I): 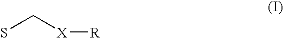
in which,
S is a monosaccharide or a polysaccharide containing up to 20 sugar units, in pyranose and/or furanose form, and of the L and/or D configuration, said mono- or polysaccharide having at least one hydroxyl function that is necessarily free and/or optionally one or more optionally protected amine functions;
the S—CH₂X bond is a bond of C-anomeric nature;
X is a group selected from: —CO—, —CH(NR₁R₂)—, —CHR′— and —C(═CHR′)—;
R is a linear or branched, saturated or unsaturated alkyl, perfluoroalkyl or hydrofluoroalkyl chain, or a cycloalkyl, cycloperfluoroalkyl or cyclohydrofluoroalkyl ring member having from 1 to 18 carbon atoms, or a phenyl radical, the said chain, said ring member or said phenyl radical optionally being interrupted with one or more heteroatoms selected from among oxygen, sulfur, nitrogen and silicon, and optionally substituted with at least one radical selected from among —OR′₁—, —SR″₁, —NR′″₁R′₂, —COOR″₂, —CONHR′″₂, —CN, halogen, perfluoroalkyl and hydrofluoroalkyl, and/or at least one optionally substituted cycloalkyl, aryl or heterocyclic radical;
R′, R₁ and R₂, which may be identical or different, have the same definition as that given for R, and can also represent a hydrogen and a hydroxyl radical;
R′₂ and R′″₂, which may be identical or different, represent a hydrogen atom, or a radical selected from among a hydroxyl radical and a linear or branched, saturated or unsaturated alkyl, perfluoroalkyl and/or hydrofluoroalkyl radical having from 1 to 20 carbon atoms;
R′₁, R″₁, R″₂ and R′″₁, which may be identical or different, represent a hydrogen atom, or a radical selected from among a linear or branched, saturated or unsaturated alkyl, perfluoroalkyl and/or hydrofluoroalkyl radical having from 1 to 20 carbon atoms;
with the following provisos:
R₁ and R₂ cannot simultaneously each be a hydroxyl radical;
R′″₁ and R′₂ cannot simultaneously each be a hydroxyl radical;
when S is a D-xylose and X is ═CO, then R cannot be an unsubstituted phenyl;
when S is a D-glucose and X is the function —OH, then R cannot be a p-hydroxyphenyl; and
when S is a D-glucose and X is ═O, then R cannot be a p-methoxyphenyl or a hexyl.

8. The regime or regimen as defined by claim 7, for preventing and/or treating a cutaneous allergic reaction, atopic dermatitis or atopic eczema.

9. The regime or regimen as defined by claim 7, for preventing and/or treating delayed contact hypersensitivity, psoriasis, vitiligo, diffuse scleroderma, lupus erythematous or alopecia.

10. The regime or regimen as defined by claim 1, said at least one compound of general formula (I) being formulated into a cosmetic/pharmaceutical composition which comprises a topically applicable, physiologically acceptable medium, and topically applied onto the skin, the mucous membranes and/or the scalp of such individual.

11. The regime or regimen as defined by claim 1, wherein general formula (I) S is a monosaccharide or a polysaccharide having up to 5 sugar units, in pyranose and/or furanose form, and of the L and/or D configuration, said mono- or polysaccharide having at least one hydroxyl function that is necessarily free and/or optionally one or more optionally protected amine functions.

12. The regime or regimen as defined by claim 1, wherein general formula (I) S is a polysaccharide having up to 6 sugar units and selected from the group consisting of D-maltose, D-cellobiose, D-maltotriose, a disaccharide combining a uronic acid selected from among D-iduronic acid or D-glucuronic acid, with a hexosamine selected from among D-galactosamine, D-glucosamine, N-acetyl-D-galactosamine and N-acetyl-D-glucosamine, or an oligosaccharide selected from the group consisting of xylobiose, methyl-β-xylobioside, xylotriose, xylotetraose, xylopentaose and xylohexaose.

13. The regime or regimen as defined by claim 1, wherein general formula (I) S is a monosaccharide or a polysaccharide having up to 2 sugar units, in pyranose and/or furanose form, and of the L and/or D configuration, said mono- or polysaccharide having at least one hydroxyl function that is necessarily free and/or optionally one or more optionally protected amine functions.

14. The regime or regimen as defined by claim 13, wherein general formula (I) S is a monosaccharide selected from the group consisting of D-glucose, D-mannose, D-xylose, D-lyxose, L-arabinose, L-rhamnose, D-glucuronic acid, D-galacturonic acid, D-iduronic acid, N-acetyl-D-glucosamine, N-acetyl-D-galactosamine, and L-fucose.

15. The regime or regimen as defined by claim 1, wherein general formula (I) R is a linear or branched, saturated or unsaturated alkyl chain, or a cycloalkyl ring member having from 1 to 14 carbon atoms, or a phenyl radical, with the proviso that said chain, said ring member or said phenyl radical may optionally be substituted with at least one radical selected from among —OR′₁, —NR′″₁R′₂, —COOR″₂ and —CONHR′″₂.

16. The regime or regimen as defined by claim 1, said at least one compound of general formula (I) being selected from the group consisting of:
Compound 1. 1-(C-β-D-Xylopyranosyl)propan-2-one; and
Compound 2. 1-(C-β-D-Glucopyranosyl)propan-2-one.

17. A regime or regimen for stimulating the immune system of the skin and/or immunoregulating same, comprising administering to an individual in need of such treatment, a thus effective amount of at least one compound having the following general formula (I): 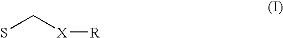
in which,
S is a monosaccharide or a polysaccharide containing up to 20 sugar units, in pyranose and/or furanose form, and of the L and/or D configuration, said mono- or polysaccharide having at least one hydroxyl function that is necessarily free and/or optionally one or more optionally protected amine functions;
the S—CH₂X bond is a bond of C-anomeric nature;
X is a group selected from: —CO—, —CH(NR₁R₂)—, —CHR′— and —C(═CHR′)—;
R is a linear or branched, saturated or unsaturated alkyl, perfluoroalkyl or hydrofluoroalkyl chain, or a cycloalkyl, cycloperfluoroalkyl or cyclohydrofluoroalkyl ring member having from 1 to 18 carbon atoms, or a phenyl radical, the said chain, said ring member or said phenyl radical optionally being interrupted with one or more heteroatoms selected from among oxygen, sulfur, nitrogen and silicon, and optionally substituted with at least one radical selected from among —OR′₁—, —SR″₁, —NR′″₁R′₂, —COOR″₂, —CONHR′″₂, —CN, halogen, perfluoroalkyl and hydrofluoroalkyl, and/or at least one optionally substituted cycloalkyl, aryl or heterocyclic radical;
R′, R₁ and R₂, which may be identical or different, have the same definition as that given for R, and can also represent a hydrogen and a hydroxyl radical;
R′₂ and R′″₂, which may be identical or different, represent a hydrogen atom, or a radical selected from among a hydroxyl radical and a linear or branched, saturated or unsaturated alkyl, perfluoroalkyl and/or hydrofluoroalkyl radical having from 1 to 20 carbon atoms;
R′₁, R″₁, R″₂ and R′″₁, which may be identical or different, represent a hydrogen atom, or a radical selected from among a linear or branched, saturated or unsaturated alkyl, perfluoroalkyl and/or hydrofluoroalkyl radical having from 1 to 20 carbon atoms;
with the following provisos:
R₁ and R₂ cannot simultaneously each be a hydroxyl radical;
R′″₁ and R′₂ cannot simultaneously each be a hydroxyl radical;
when S is a D-xylose and X is ═CO, then R cannot be an unsubstituted phenyl;
when S is a D-glucose and X is the function —OH, then R cannot be a p-hydroxyphenyl; and
when S is a D-glucose and X is ═O, then R cannot be a p-methoxyphenyl or a hexyl.

18. A composition comprising at least one compound having the general formula (I) as defined in claim 1 and at least one active agent for the hair, formulated into a physiologically acceptable medium therefor.

19. The composition as defined by claim 18, said at least one active agent for the hair being selected from the group consisting of:
anti-seborrhoeic agents;
agents for combating squamous states of the scalp;
active agents for stimulating hair regrowth and/or promoting the slowing down of hair loss;
antibiotics;
cinnarizine, nimodipine and nifedipine;
hormones;
anti-androgenic agents;
cromakalim and nicrorandil.

20. The composition as defined by claim 19, said at least one active agent for the hair being selected from the group consisting of:
sulfur-containing amino acids, 13-cis-retinoic acid or cyproterone acetate;
zinc pyrithione, selenium disulfide, climbazole, undecylenic acid, ketoconazole, piroctone olamine (octopirox) or cyclopiroctone (cyclopirox);
nicotinic acid esters, tocopheryl nicotinate, benzyl nicotinate and C₁-C₆ alkyl nicotinates;
pyrimidine derivatives, 2,4-diamino-6-piperidinopyrimidine 3-oxide or “Minoxidil”; Aminexil or 2,4-diaminopyrimidine 3-oxide;
agents that are both lipoxygenase inhibitors and cyclooxygenase inducers, or cyclooxygenase-inducing agents that promote hair regrowth;
macrolides, pyranosides, tetracyclines and erythromycin;
estriol, or thyroxine and salts thereof; and
oxendolone, spironolactone, diethylstilbestrol and flutamide.
